(19) United States
(12) Patent Application Publication
(10) Pub. No.: US 2024/0016722 A1
(43) Pub. Date: Jan. 18, 2024

(54) MASCARA COMPOSITION AND METHOD OF MAKING

(71) Applicant: Coty Inc., New York, NY (US)

(72) Inventor: Tamar Lesnoy, Fair Lawn, NJ (US)

(21) Appl. No.: 18/255,526

(22) PCT Filed: Dec. 1, 2021
(86) PCT No.: PCT/US2021/061461

§ 371 Date: Jun. 1, 2023

Related U.S. Application Data
(60) Provisional application No. 63/121,577, filed on Dec. 4, 2020.

(51) Int. Cl. A61K 8/81; A61Q 1/10; A61K 8/92; A61K 8/73; A61K 8/19

(52) U.S. Cl. CPC A61K 8/8176; A61Q 1/10; A61K 8/8147; A61K 8/927; A61K 8/731; A61K 8/19; A61K 2800/43; A61K 2800/594

(57) ABSTRACT

Various embodiments disclosed relate to a mascara composition. The mascara composition includes a polyacrylate component. The mascara composition further optionally includes at least one of a polyvinylpryrrollidone component, an acrylate copolymer component, an acrylic acid copolymer component, an eicosene copolymer component, a structurant component, and a fiber component.

### RELATED APPLICATION

This Application claims priority to U.S. Provisional Patent Application No. 63/121,577, titled MASCARA COMPOSITION AND METHOD OF MAKING, filed on Dec. 4, 2020, the contents of which are hereby incorporated by reference.

### BACKGROUND

Mascara compositions can provide desirable appearance to a user's eyelash. For example, a mascara composition can help to make an eyelash appear longer and more voluminous. There is a need to improve compositions to enhance these properties.

### SUMMARY OF THE INVENTION

Various aspects disclosed relate to a mascara composition. The mascara composition includes a polyacrylate component. Optional components include a polyvinylpryrrollidone component, an acrylate copolymer component, an acrylic acid copolymer component, an eicosene copolymer component, a structurant component, and a fiber component.

Various aspects disclosed relate to a kit. The kit comprises a vessel. The kit further comprises a mascara composition disposed within the vessel. The mascara composition includes a polyacrylate component. Optional components include a polyvinylpryrrollidone component, an acrylate copolymer component, an acrylic acid copolymer component, an eicosene copolymer component, a structurant component, and a fiber component. The kit further includes an applicator in contact with at least a portion of the mascara composition.

Various aspects disclosed further relate to a method of making a mascara composition. The method includes processing a polyacrylate component and optionally at least one of a polyvinylpryrrollidone component, an acrylate copolymer component, an acrylic acid copolymer component, an eicosene copolymer component, a structurant component, and a fiber component.

### BRIEF DESCRIPTION OF THE FIGURES

The drawings illustrate generally, by way of example, but not by way of limitation, various embodiments of the present invention.

FIG. 1 is a line graph showing perceived dryness of Mascara Composition 1, in accordance with various embodiments.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

The instant disclosure relates to a mascara formulation. The mascara formulation is capable of increasing the length, lift, volume, lash extension effect, or a combination thereof of an eyelash to which it is applied. Moreover, the mascara formulation is capable of drying relatively rapidly following application.

The benefits of the instantly described mascara formulations can be achieved with the inclusion of several components. Examples of these components include a polyacrylate component. The mascara optionally includes at least one of a polyvinylpryrrollidone component, an acrylate copolymer component, an acrylic acid copolymer component, an eicosene copolymer component, a structurant component, a fiber component, or a mixture thereof.

Where present, the polyacrylate component is in a range of from about 0.1 wt % to about 1 wt % of the mascara composition, about 0.25 wt % to about 0.35 wt %, less than, equal to, or greater than about 0.1 wt %, 0.2, 0.3, 0.4, 0.5, 0.6, 0.7, 0.8, 0.9, or about 1 wt %/o. Where present, the polyvinylpryrrollidone component is in a range of from about 1 wt % to about 5 wt % of the mascara composition, about 1.5 wt % to about 2.5 wt %, less than, equal to, or greater than about 1.5 wt %, 1.6, 1.7, 1.8, 1.9, 2.0, 2.1, 2.2, 2.3, 2.4, or about 2.5. Where present, the acrylate copolymer component is in a range of from about 0.5 wt % to about 4 wt % of the mascara composition about 1.5 wt % to about 2.5 wt %, less than, equal to, or greater than about 0.5 wt %, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or about 4.0 wt %. Where present, the acrylic acid copolymer component is in a range of from about 0.2 wt % to about 5 wt % of the mascara composition about 0.5 wt % to about 1.5 wt %, less than, equal to, or greater than about 0.2 wt %, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5 wt %. Where present, the eicosene copolymer component is in a range of from about 0.5 wt % to about 3.5 wt % of the mascara composition about 1.5 wt % to about 2.5 wt %, less than, equal to, or greater than about 0.5 wt %, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or about 3.5 wt %. Where present, the structurant component is in a range of from about 1 wt % to about 50 wt % of the mascara composition about 4 wt % to about 8 wt %, less than, equal to, or greater than about 1 wt %, 5, 10, 15, 20, 25, 30, 35, 40, 45, or about 50 wt %. Where present, the fiber component is in a range of from about 0.1 wt % to about 0.5 wt %, 0.2 wt % to about 0.4 wt %, less than, equal to, or greater than about 0.1 wt %, 0.2, 0.3, 0.4, or 0.5 wt % of the mascara composition.

Where present, the polyacrylate component is a mixture of sodium polyacrylate and acrylic acid. An example of a sodium polyacrylate is available under the trade designation COVARYL AC, available from Sensient Cosmetic Technologies, Milwuakee WI. COVARYL AC is a polyacrylic acid, sodium salt and water soluble thickening agent. It is a high purity acrylic resin, stringy clear gel that exhibits no greasy or tacky feeling. In the mascara formulation, COVARYL AC provides stretchy texture and overcomes the difficulties associated with using other polymers that don't allow for good processing or solubility. Where present, the acrylate copolymer component includes a mixture of an ammonium acrylate copolymer, water, alcohol denat., disodium deceth-6 sulfosuccinate, laureth-30, and sodium dehydroacetate. Where present, the acrylic acid copolymer component includes a mixture of water, ethylene/acrylic acid copolymer, styrene/acrylates copolymer, C11-15 pareth-7, C11-15 pareth 40, phenoxyethanol, caprylyl glycol, and sodium laureth-12 sulfate. Where present, the eicosene copolymer includes an alkylated vinyl pyrrolidone. Where present, the structurant component includes a paraffin wax, a beeswax, a caranuba wax, or a mixture thereof. Although waxes are mentioned as structurants, further structurants can include any other suitable solid component. Where present, the fiber component includes a polyamide fiber, a cellulose fiber, or a mixture thereof. If the fiber component includes a polyamide fiber, a length of an individual polyamide fiber can be in a range of from about 1 mm to about 5 mm, about 2 mm to about 4 mm, less than, equal to, or greater than about 1 mm, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mm. Any wax or fiber included can be a natural or synthetic product or there can be a mixture of natural and synthetic products. Where present, the polyvinylpryrrollidone component includes a PVP K-30 polymer having a molecular weight in a range of from about 40,000 g/mol to about 80,000 g/mol, about 50,000 g/mol to about 70 g/mol, less than, equal to, or greater than about 40,000 g/mol, 45,000, 50,000, 55,000, 60,000, 65,000, 70,000, 75,000, or about 80,000 g/mol. It was found that PVP K-30 yielded unexpected results even over other polyvinylpryrrollidone components such as PVP K-90, which yielded an extremely thick, goopy, tacky mascara formulation.

The mascara composition can include further auxiliary components, carrier substances, active agents or mixtures thereof. Examples include preservatives, colorings, pigments with coloring effect, thickeners, fragrances, alcohols, polyols, electrolytes, gelling substances, polar and nonpolar oils, further polymers and/or copolymers, emulsifiers, waxes, stabilizers, antioxidants, humectants, or mixtures thereof.

Examples of pigments, pigment mixtures or powders with a pigment-like effect, also including those with a pearl-gloss effect, may include, for example, iron oxides, chromium, oxides, aluminum silicates such as ochre, titanium (di)oxide, mica, kaolin, manganese containing clays such as umber and red bole, calcium carbonate, French chalk, mica-titanium oxide, mica-titanium oxide-iron oxide, bismuth oxychloride, nylon beads, ceramic beads, expanded and non-expanded synthetic polymer powders, cellulose/rayon, teflon, cotton fibers, powdery natural organic compounds such as milled solid algae, milled plant parts, blue #1, carmine, yellow #5, encapsulated and non-encapsulated cereal starches and mica-titanium oxide-organic dye. Where present, the pigments can be in a range of from about 5 wt % to about 15% of the mascara composition, about 8 wt % to about 13 wt %, less than, equal to, or greater than about 5 wt %, 5.5, 6, 6.5, 7, 7.5, 8, 8.5, 9, 9.5, 10, 10.5, 11, 11.5, 12, 12.5, 13, 13.5, 14, 14.5, or about 15 wt %.

Examples of antioxidants include vitamins such as vitamin C and derivatives thereof, for example, ascorbic acetate, ascorbic phosphate, and ascorbic palmitate; vitamin A and derivatives thereof; folic acid and derivatives thereof; vitamin E and derivatives thereof, such as tocopheryl acetate; flavones or flavonoids; amino acids, such as histidine, glycine, tyrosine, tryptophan, and derivatives thereof; carotenoids and carotenes such as, for example, α-carotene, β-carotene; uric acid and derivatives thereof; α-hydroxy acids such as citric acid, lactic acid, malic acid; stilbene and derivatives thereof. Where present, the antioxidant component can be in a range of from about 0.1 wt % to about 2 wt % of the mascara composition, about 0.5 wt % to about 1 wt %, less than, equal to, or greater than about 0.1 wt %, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or about 2 wt % of the mascara composition.

Examples of a conditioning agent can include vitamin E. Where present, the conditioning agent can be in a range of from about 0.1 wt % to about 2 wt % of the mascara composition, about 0.5 wt % to about 1 wt %, less than, equal to 0.1 wt %, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or about 2.0 wt %.

Examples of a humectant can include propylene glycol, butylene glycol, glycerol, or a mixture thereof.

Mascaras with the composition according to the invention may exist as O/W or W/O emulsions. Suitable emulsifiers for O/W emulsions are for instance addition products of 2-30 mol ethylene oxide to linear C₈-C₂₂ fatty alcohols, to C₁₂-C₂₂ fatty acids and to C₈-C₁₅ alkylphenols; C₁₂-C₂₂ fatty acid monoesters and diesters of addition products of 1-30 mol ethylene oxide to glycerine.

For W/O emulsions it is preferred to incorporate the emulsifier at a different step after the above mentioned complex is formed. Copolymers of polysiloxane polyalkyl polyethers are possible emulsifiers. A suitable emulsifier is Cetyl Dimethicone Copolyol.

In some examples, the mascara composition can include the components listed in Table 1. As shown in Table 1, the mascara composition includes a plurality of stages that are ultimately mixed together to form the mascara composition. Wt % ranges listed in Table 1 for each component are relative to the entire mascara composition.

TABLE 1

| | |
|---|---|
| Material | Wt % |

Stage 1

| | |
|---|---|
| White Beeswax | 0.5-1.0 |
| UNIMER U-15 (alkylated vinylpyrrolidone | 1-3 |
| copolymer) | |
| CITHROL GMS 40-PW-(GD) (Glyceryl | 5-6 |
| Monostearate) | |
| Paraffin | 4-5 |
| 2442 Carnauba Wax T1 | 2-4 |
| Hystrene 5016 NF Flake Veg (10555) (a triple- | 1-3 |
| pressed stearic acid vegetable in flake form) | |
| Bentone Gel CAO V (a specially prepared | 0.25-0.75 |
| dispersion of a non-animal organically | |
| modified hectorite in cosmetic grade castor | |
| oil) | |
| Protachem LL (L-Lysine derivative) | 0.25-0.75 |
| Caprylic/Capric Triglyceride | 0.5-1.5 |
| Gransil VX-418 (wax comprised of a short | 0.2-0.6 |
| chain silicone and C18 stearyl group) | |

Stage 2

| | |
|---|---|
| Deionized Water | 50-57 |
| Disodium EDTA | 0.05-0.15 |
| L-Arginine C | 0.5-1.5 |

Stage 3

| | |
|---|---|
| SunCROMA Black Iron Oxide C33-5000 | 2-6 |
| SunCROMA Black Iron Oxide C33-6000 | 1-5 |
| Orgasol 4000 EX D Nat Cos Caresse | 0.1-0.5 |
| (Azacyclotridecan-2-one, polymer with | |
| hexahydro-2H-azepin-2-one) | |

Stage 4

| | |
|---|---|
| Tween 20-LQ-(AP) (Polyoxyethylene | 0.8-2.2 |
| sorbitan monolaurate) | |

Stage 5

| | |
|---|---|
| Propylene Glycol USP | 1-5 |
| Natrosol 250 HHR PC | 0.5-1 |
| (Hydroxyethylcellulose) | |

Stage 6

| | |
|---|---|
| PVP (Polyvinylpyrrolidone) | 1-6 |

Stage 7

| | |
|---|---|
| PVP (Polyvinylpyrrolidone) | 0.5-1 |

Stage 8

| | |
|---|---|
| Propylene Glycol USP | 0.5-1.5 |
| Covacryl AC (61241) (polyacrylic acid, | 0.1-0.5 |
| sodium salt and water soluble thickening | |
| agent) | |

Stage 9

| | |
|---|---|
| Syntran PC 5288 (an amorphous functional | 0.5-1.5 |
| acrylic polymer section grafted onto a | |
| crystalline polyethylene backbone) | |

Stage 10

| | |
|---|---|
| Deionized Water | 1-5 |
| Worlée Micromer C60/42 (an acrylic | 1-4 |
| copolymer) | |
| NFBL-10D-2T (a black Nylon fiber in short | 0.05-0.5 |
| cut form, composed of Nylon 6 (And) Iron | |
| Oxides (C.I. 77499) (And) | |
| Triethoxycaprylylsilane (And) Silica) | |
| NFCB 17D 4T (a black Nylon fiber in short | 0.05-0.5 |
| cut form, composed of Nylon-6 and Black 2) | |
| Cell-U-Lash 150 (an odorless, fine white | 0.05-0.5 |
| powder used for CELL-U-LASH ™ fibers. | |
| CELL-U-LASH ™ fibers are made of | |
| cellulose and vary in length from 20 μm to | |
| 110 μm. CELL-U-LASH ™ 150 is 80-110 | |
| micrometers) | |

Stage 11

| | |
|---|---|
| Euxyl PE 9010 (a liquid cosmetic | 0.6-1 |
| preservative based on phenoxyethanol and | |
| ethylhexylglycerin) | |
| WATER (and) PANTHENOL (and) | 0.6-1 |
| PANTOLACTONE | |
| VITAMIN E ACETATE | 0.05-0.4 |

An additional example of a suitable mascara composition is shown in Table 2. The formulation of Table 2 differs from that of Table 1 in that some materials that are “plastic” are removed and replaced with more biodegradable materials such as Organsol 4000 EX™.

TABLE 2

| | |
|---|---|
| Material | Wt % |

Stage 1

| | |
|---|---|
| Candelilla Wax | 0.5-1.0 |
| Sunflower | 3-8 |
| UNIMER U-15 (alkylated vinylpyrrolidone | 1-3 |
| copolymer) | |
| Kesterwax K60P - Polyhydroxystearic acid | 0.5-2 |
| CITHROL GMS 40-PW-(GD) (Glyceryl | 5-6 |
| Monostearate) | |
| 2442 Carnauba Wax T1 | 2-4 |
| Hystrene 5016 NF Flake Veg (10555) (a triple- | 1-3 |
| pressed stearic acid vegetable in flake form) | |
| Kahlwax 6290 - Rhus Verniciflua Peel Cera/Rhus | 0.1-1 |
| Verniciflua Peel Wax | |
| Bentone Gel CAO V (a specially prepared dispersion | 0.25-0.75 |
| of a non-animal organically modified hectorite in | |
| cosmetic grade castor oil) | |
| Protachem LL (L-Lysine derivative) | 0.25-0.75 |
| Caprylic/Capric Triglyceride | 0.5-1.5 |
| Gransil VX-418 (wax comprised of a short chain | 0.2-0.6 |
| silicone and C18 stearyl group) | |

Stage 2

| | |
|---|---|
| Deionized Water | 50-57 |
| Disodium EDTA | 0.05-0.15 |
| L-Arginine C | 0.5-1.5 |

Stage 3

| | |
|---|---|
| SunCROMA Black Iron Oxide C33-5000 | 2-6 |
| SunCROMA Black Iron Oxide C33-6000 | 1-5 |

Stage 4

| | |
|---|---|
| Tween 20-LQ-(AP) (Polyoxyethylene sorbitan | 0.8-2.2 |
| monolaurate) | |

Stage 5

| | |
|---|---|
| Propylene Glycol USP | 1-5 |
| Natrosol 250 HHR PC (Hydroxyethylcellulose) | 0.5-1 |

Stage 6

| | |
|---|---|
| PVP (Polyvinylpyrrolidone) | 1-6 |

Stage 7

| | |
|---|---|
| PVP (Polyvinylpyrrolidone) | 0.5-1 |

Stage 8

| | |
|---|---|
| Propylene Glycol USP | 0.5-1.5 |
| Covacryl AC (61241) (polyacrylic acid, sodium salt | 0.1-0.5 |
| and water soluble thickening agent) | |

Stage 9

| | |
|---|---|
| Deionized Water | 1-5 |
| Worlée Micromer C60/42 (an acrylic copolymer) | 3-12 |
| RAYON FIBER 3.3T-0.8MM(S) - Rayon (and) | 0.01-0.5 |
| Silica | |
| Cell-U-Lash 150 (an odorless, fine white powder | 0.05-0.5 |
| used for CELL-U-LASH ™ fibers. CELL-U-LASH ™ | |
| fibers are made of cellulose and vary in length from | |
| 20 μm to 110 μm. CELL-U-LASH ™ 150 is 80-110 | |
| micrometers) | |

Stage 10

| | |
|---|---|
| Spectrastat - Caprylhydroxamic Acid (and) Caprylyl | 0.6-1 |
| Glycol (and glycerin) | |
| WATER (and) PANTHENOL (and) | 0.6-1 |
| PANTOLACTONE | |
| VITAMIN E ACETATE | 0.05-0.4 |

The mascara compositions described herein are capable of rapid drying. For example, the mascara composition can dry within a range of from about 1 minute to about 10 minutes following application to an eyelash, about 3 minutes to about 7 minutes, less than, equal to, or greater than about 1 minute, 2, 3, 4, 5, 6, 7, 8, 9, or about 10 minutes. Additionally, the mascara composition can visibly lengthen the appearance of an eyelash in a range of from about 20% to about 200%, relative to a baseline eyelash that is free of the mascara, about 30% to about 50%, less than, equal to, or greater than about 20%, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or about 200%. Further, the mascara composition can visibly increase the volume of an eyelash in a range of from about 5× to about 15×, relative to a baseline eyelash that is free of the mascara, about 6× to about 10×, less than, equal to, or greater than about 5×, 6×, 7×, 8×, 9×, 10×, 11×, 12×, 13×, 14×, or about 15×.

The mascara composition can be a component of a mascara kit. The mascara kit can include a vessel into which the mascara composition is deposited. The kit can further include an applicator such as a brush or wand that is removably disposed at least partially within the vessel. The brush or wand can have a micro brush head, a bubble brush head, a pointed hourglass cone head, an oversized head, a cone taper head, a curved head, a balled tip head, a microwand head, a tuber shorty head, or a flat comb head.

The mascara composition can be made according to any suitable method. According to some examples, the mascara composition can be formed by processing the polyacrylate component, the polyvinylpryrrollidone component, the acrylate copolymer component, the acrylic acid copolymer component, the eicosene copolymer component, the structurant component, and the fiber component.

According to further embodiments, individual stages (such as those identified in Table 1) can be individually fed into a homogenizer to ultimately form the mascara composition. Individual stages may be heated to a temperature suitable to bring several or all components of the stage to a liquid phase before being fed into the homogenizer. The environment within the homogenizer can be kept at a predetermined temperature or the temperature can vary. The homogenizer may include a rotating element such as a blade or paddle to mix the components. The degree to which the rotating element rotates can be controlled to be constant or variable during the course of manufacturing the mascara composition.

### EXAMPLES

Various embodiments of the present invention can be better understood by reference to the following Examples which are offered by way of illustration. The present invention is not limited to the Examples given herein.

Various properties of the described mascara composition were determined according to the below Example.

### Materials

Mascaras tested included Composition 1, Composition 2, Comparative Composition 1, and Comparative Composition 2.

TABLE 3
Composition 1

| | |
|---|---|
| Material | Wt % |

Stage 1

| | |
|---|---|
| White Beeswax | 0.5-1.0 |
| UNIMER U-15 (alkylated vinylpyrrolidone | 1-3 |
| copolymer) | |
| CITHROL GMS 40-PW-(GD) (Glyceryl | 5-6 |
| Monostearate) | |
| Paraffin | 4-5 |
| 2442 Carnauba Wax T1 | 2-4 |
| Hystrene 5016 NF Flake Veg (10555) | 1-3 |
| Bentone Gel CAO V | 0.25-0.75 |
| Protachem LL (L-Lysine derivative) | 0.25-0.75 |
| Caprylic/Capric Triglyceride | 0.5-1.5 |
| Gransil VX-418 (wax comprised of a short | 0.2-0.6 |
| chain silicone and C18 stearyl group) | |

Stage 2

| | |
|---|---|
| Deionized Water | 50-57 |
| Disodium EDTA | 0.05-0.15 |
| L-Arginine C | 0.5-1.5 |

Stage 3

| | |
|---|---|
| SunCROMA Black Iron Oxide C33-5000 | 2-6 |
| SunCROMA Black Iron Oxide C33-6000 | 1-5 |
| Orgasol 4000 EX D Nat Cos Caresse | 0.1-0.5 |
| (Azacyclotridecan-2-one, polymer with | |
| hexahydro-2H-azepin-2-one) | |

Stage 4

| | |
|---|---|
| Tween 20-LQ-(AP) | 0.8-2.2 |

Stage 5

| | |
|---|---|
| Propylene Glycol USP | 1-5 |
| Natrosol 250 HHR PC | 0.5-1 |
| (Hydroxyethylcellulose) | |

Stage 6

| | |
|---|---|
| PVP (Polyvinylpyrrolidone) | 1-6 |

Stage 7

| | |
|---|---|
| PVP (Polyvinylpyrrolidone) | 0.5-1 |

Stage 8

| | |
|---|---|
| Propylene Glycol USP | 0.5-1.5 |
| Covacryl AC (61241) (polyacrylic acid, | 0.1-0.5 |
| sodium salt and water soluble thickening | |
| agent) | |

Stage 9

| | |
|---|---|
| Syntran PC 5288 (an amorphous functional | 0.5-1.5 |
| acrylic polymer section grafted onto a | |
| crystalline polyethylene backbone) | |

Stage 10

| | |
|---|---|
| Deionized Water | 1-5 |
| Worlée Micromer C60/42 (an acrylic | 1-4 |
| copolymer) | |
| NFBL-10D-2T (a black Nylon fiber in short | 0.05-0.5 |
| cut form, composed of Nylon 6 (And) Iron | |
| Oxides (C.I. 77499) (And) | |
| Triethoxycaprylylsilane (And) Silica) | |
| NFCB 17D 4T (a black Nylon fiber in short | 0.05-0.5 |
| cut form, composed of Nylon-6 and Black 2) | |
| Cell-U-Lash 150 (an odorless, fine white | 0.05-0.5 |
| powder used for CELL-U-LASH ™ fibers. | |
| CELL-U-LASH ™ fibers are made of | |
| cellulose and vary in length from 20 μm to | |
| 110 μm. CELL-U-LASH ™ 150 is 80-110 | |
| micrometers) | |

Stage 11

| | |
|---|---|
| Euxyl PE 9010 (a liquid cosmetic | 0.6-1 |
| preservative based on phenoxyethanol and | |
| ethylhexylglycerin) | |
| WATER (and) PANTHENOL (and) | 0.6-1 |
| PANTOLACTONE | |
| VITAMIN E ACETATE | 0.05-0.4 |

TABLE 4
Composition 2

| | |
|---|---|
| Material | Wt% |

Stage 1

| | |
|---|---|
| Sunflower wax | 3-8 |
| Candelilla wax | 0.5-1.0 |
| UNIMER U-15 (alkylated vinylpyrrolidone | 1-3 |
| copolymer) | |
| Kesterwax K60P - Polyhydroxystearic acid | 0.5-2 |
| CITHROL GMS 40-PW-(GD) (Glyceryl | 5-6 |
| Monostearate) | |
| 2442 Carnauba Wax T1 | 2-4 |
| Hystrene 5016 NF Flake Veg (10555) (a triple- | 1-3 |
| pressed stearic acid vegetable in flake form) | |
| Kahlwax 6290 - Rhus Verniciflua Peel Cera/Rhus | 0.1-1 |
| Verniciflua Peel Wax | |
| Bentone Gel CAO V (a specially prepared dispersion | 0.25-0.75 |
| of a non-animal organically modified hectorite in | |
| cosmetic grade castor oil) | |
| Protachem LL (L-Lysine derivative) | 0.25-0.75 |
| Caprylic/Capric Triglyceride | 0.5-1.5 |
| Gransil VX-418 (wax comprised of a short chain | 0.2-0.6 |
| silicone and C18 stearyl group) | |

Stage 2

| | |
|---|---|
| Deionized Water | 50-57 |
| Disodium EDTA | 0.05-0.15 |
| L-Arginine C | 0.5-1.5 |

Stage 3

| | |
|---|---|
| SunCROMA Black Iron Oxide C33-5000 | 2-6 |
| SunCROMA Black Iron Oxide C33-6000 | 1-5 |

Stage 4

| | |
|---|---|
| Tween 20-LQ-(AP) (Polyoxyethylene sorbitan | 0.8-2.2 |
| monolaurate) | |

Stage 5

| | |
|---|---|
| Propylene Glycol USP | 1-5 |
| Natrosol 250 HHR PC (Hydroxyethylcellulose) | 0.5-1 |

Stage 6

| | |
|---|---|
| PVP (Polyvinylpyrrolidone) | 1-6 |

Stage 7

| | |
|---|---|
| PVP (Polyvinylpyrrolidone) | 0.5-1 |

Stage 8

| | |
|---|---|
| Propylene Glycol USP | 0.5-1.5 |
| Covacryl AC (61241) (polyacrylic acid, sodium salt | 0.1-0.5 |
| and water soluble thickening agent) | |

Stage 9

| | |
|---|---|
| Deionized Water | 1-5 |
| Worlée Micromer C60/42 (an acrylic copolymer) | 3-12 |
| RAYON FIBER 3.3T-0.8MM(S) - Rayon (and) | 0.01-0.5 |
| Silica | |
| Cell-U-Lash 150 (an odorless, fine white powder | 0.05-0.5 |
| used for CELL-U-LASH ™ fibers. CELL-U-LASH ™ | |
| fibers are made of cellulose and vary in length from | |
| 20 μm to 110 μm. CELL-U-LASH ™ 150 is 80-110 | |
| micrometers) | |

Stage 10

| | |
|---|---|
| Spectrastat - Caprylhydroxamic Acid (and) Caprylyl | 0.6-1 |
| Glycol (and glycerin) | |
| WATER (and) PANTHENOL (and) | 0.6-1 |
| PANTOLACTONE | |
| VITAMIN E ACETATE | 0.05-0.4 |

TABLE 5
Constituents of Comparative Formula 1

G683939 16 aqua/water/eau
Propylene glycol
Styrene/acylates/ammonium methacrylate copolymer
Polyurethane-35
Cera alba/beeswax/cire dabeille
Synthetic fluorphlogopite
Glyceryl stearate
Cetyl alcohol
PEG-200 glyceryl stearate
Ethylenediamine/stearyl dimer dilinoleate copolymer
Copernica cerifera cera/carnauba wax/cire de carnauba
Stearoc acid
Palmitic acid
Ethylne/va copolymer
Alcohol denat.
Paraffin
Aminomethyl propanediol
Glycerin
hydroxyethylcellulose
phenoxyethanol
Caprylyl glycol
Butylene glycol
xanthan gum
Sodium laureth sulfate
Disodium EDTA
Tetrasodium EDTA
Pentaeythrityl tetra-di-t-butyl hydroxyhydrocinna-mate
Silica
Iron oxide
Titanium dioxide
Mica
Hromium oxide

TABLE 6
Constituents of Comparative Formula 2

Water
EAU/paraffin/cera/alba
Beeswax
Cire d'abeille
Stearic acid
Cara carnauba
Carnauba wax
ACACIA Senegal Gum
Palmitic acid
Triethanolamine
Hydroxyethlcellulose
Aminomethyl
Propanediol
PEG-40 sterate
Sodium polymethacrylate
Methylparaben
Propylparaben
Hydrogenate jojoba oil
Hydrogenated palm oil
Simethicone
BHT
Polyquaternium-10
Panthenol
Iron oxide
Chromium oxide
Titanium dioxide

Three different mascara wands were used to apply the respective mascara compositions to an individual tester's eyelash. The brushes are named “Mini Cashmere”, “Burgundy”, and “Grey Flower”. Details on each wand are provided below in Table 4. Comparative Compositions 1 and 2 used a hard plastic brush.

| Name | Mini Cashmere | Burgundy | Grey Flower |
|---|---|---|---|
| Shape | Skinny micro | Bubble | Pointed |
| | brush | brush | Hourglass Cone |

To test the mascara compositions, a sensory panel was established. The sensory panel included a total of 11 trained expert female panelists. The ages ranged from 38-68 years old. Each panelist had 3 years of experience in evaluating beauty care products.

### Test Methodology

Descriptive analysis approach was used to understand various mascara sensory attributes and the degree to which these attributes vary between the mascara samples. Panelists were given a training on various mascara sensory attributes (Initial texture, brush load, application and lash feel). An attribute list was generated after consulting with the project lead team member and the list was further discussed with panelists for attribute concept alignment and consensus. Attribute intensity was measured on a 0(None)-10(High) continuous linear line scale. Samples were presented in sequential monadic pattern over 3 sessions. In each session panelists applied one product for both eyes and evaluated various sensory attributes.

Each panelist applied the respect mascara composition with 15 strokes of the mascara compositions to top lashes of the left eye. The brush was re-dipped. Then 15 strokes of the mascara composition was applied to the top lashes on the right eye. Then the brush was re-dipped. Then 15 strokes of the mascara compositions were applied to top lashes of the left eye. The brush was re-dipped. Then 15 strokes of the mascara composition was applied to the top lashes on the right eye. The panelist then waited 5 minutes and took a picture of their eye and evaluated the result on a scale of 1-10 based on a ballot, the options of which are listed below in Table 5. Furthermore, each panelist took a picture of eyelash without having any mascara applied, this was designated as a baseline eyelash.

TABLE 7
Sensory Evaluation Properties
Property

Product Load on the Brush - The amount of product seen on the brush.
Brush Wet Look - The amount of wet look seen on the brush.
Coating Evenness on Brush - The degree to which product coated evenly on the brush.
Globs on the Brush - The amount of globs/chunks seen on the brush/bristles
Amount of Build - The amount of product deposited on lash after 5 strokes.
Slippery/Glide - The perception of ease in moving the brush on lashes without drag.
Ease in Application (Final) - Overall ease with which product can be applied on lashes.
Smearing - The amount of smearing observed during the application of the product
Lash Dryness @ 1 minute - The amount of dryness perceived on lashes.
Evaluate “Lash Dryness” attribute at 1 minute intervals.
Lash Dryness @ 2 minute - The amount of dryness perceived on lashes.
Lash Dryness @ 3 minute - The amount of dryness perceived on lashes.
Lash Dryness @ 4 minute - The amount of dryness perceived on lashes
Lash Dryness @ 5 minute - The amount of dryness perceived on lashes.
Lash Color Intensity - The Intensity or strength of color on lash.(darkness of lashes)
Lash Thickness - The degree to which individual lashes appears thick.
Coating Evenness on Lash - The degree to which product coating on lashes appears evenly.
Lash Volume - The degree to which lashes appear FULL on lashes (band).
Lash Lengthening Effect - The degree to which lashes appear increase in length.
Lash Look - Glossy - The amount of gloss/shine perceived on lash.
Lash Heaviness - The degree to which lash feel heavy.
Lift - The degree to which lashes appear lifted upwards from baseline to towards brow.
Curl - The degree to which lashes curl towards brow after lifting.
Lash Separation - The degree to which lash appears separate.
Lash Clumpiness - The amount of globs/clumps/particles seen on lashes.
Lash Groupings - The degree to which lashes appear in small groups.
Lash Tautness -The degree to which lashes feel stiff.(bat lashes with finger)
Transfer - The amount of smudging seen on finger when you bat lashes
Ease of Removal - Ease with which the product can be removed with a makeup remover wipe.

### Results

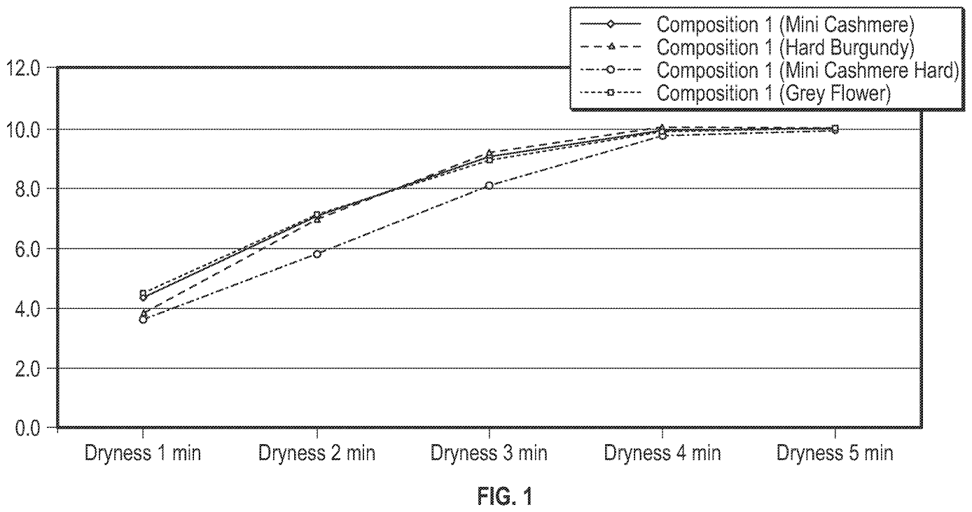

Evaluation of key sensory attributes between an untreated (baseline) eyelash as well as eyelashes treated with Composition 1, Comparative Composition 1, Comparative Composition 2, and Comparative Composition 3 are presented, in part, in FIG. 1 and in the tables below. The values listed in the tables and FIG. 1 are the average result presented by the panel. As seen in the data, Composition 1 was able to deliver substantially similar performance to Comparative Composition 1, Comparative Composition 2, and Comparative Composition 3. Thus, the data demonstrates that Composition 1 is a commercially acceptable mascara composition.

FIG. 1 is a graph showing the rate at which Composition 1 dries. As shown, Composition 1 dries a t a rapid rate.

Table 8 shows the average values assigned by the sensory panel for the lash length attribute.

TABLE 8

| | | Average |
|---|---|---|
| Composition | Brush | Value |
| Baseline (untreated eyelash) | N/A | 3.6 |
| Composition 1 | Mini Cashmere | 6.8 |
| Composition 1 | Mini Cashmere (harder grade | 7.0 |
| | of plastic by Shore value) | |
| Composition 1 | Burgundy | 7.1 |
| Composition 1 | Grey Flower | 7.0 |
| Comparative Composition 1 | Hard Plastic | 7.4 |
| Comparative Composition 2 | Hard Plastic | 7.8 |

Table 9 shows the average values assigned by the sensory panel for the lash volume attribute.

TABLE 9

| | | Average |
|---|---|---|
| Composition | Brush | Value |
| Baseline (untreated eylash) | N/A | 3.4 |
| Composition 1 | Mini Cashmere | 6.4 |
| Composition 1 | Mini Cashmere | 6.4 |
| | (harder grade) | |
| Composition 1 | Burgundy | 5.9 |
| Composition 1 | Grey Flower | 6.4 |
| Comparative Composition 1 | Hard Plastic | 7.0 |
| Comparative Composition 2 | Hard Plastic | 6.8 |

Table 10 shows the average values assigned by the sensory panel for the lash curl attribute.

TABLE 10

| | | Average |
|---|---|---|
| Composition | Brush | Value |
| Baseline (untreated eyelash) | N/A | 2.4 |
| Composition 1 | Mini Cashmere | 5.5 |
| Composition 1 | Mini Cashmere | 6.3 |
| | (harder grade) | |
| Composition 1 | Burgundy | 5.7 |
| Composition 1 | Grey Flower | 5.9 |

Table 11 shows the average values assigned by the sensory panel for the lash lift attribute.

TABLE 11

| | | Average |
|---|---|---|
| Composition | Brush | Value |
| Baseline (untreated eyelash) | N/A | 2.5 |
| Composition 1 | Mini Cashmere | 5.8 |
| Composition 1 | Mini Cashmere | 7.0 |
| | (harder grade) | |
| Composition 1 | Burgundy | 6.4 |
| Composition 1 | Grey Flower | 6.2 |

Average perceived dryness at time points following application of 1 minute, 2 minutes, 3, minutes, 4 minutes, and 5 minutes are presented in FIG. 1.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Aspects

The following exemplary aspects are provided, the numbering of which is not to be construed as designating levels of importance:
Aspect 1 provides a mascara composition comprising:
a polyacrylate component; and optionally
   a polyvinylpryrrollidone component;
   an acrylate copolymer component;
   an acrylic acid copolymer component;
   an eicosene copolymer component;
   a structurant component;
   a fiber component; or
   a mixture thereof.
Aspect 2 provides the mascara composition of Aspect 1, wherein:
the polyacrylate component is in a range of from about 0.1 wt % to about 1 wt % of the mascara composition;
the polyvinylpryrrollidone component is in a range of from about 1 wt % to about 5 wt % of the mascara composition;
the acrylate copolymer component is in a range of from about 0.5 wt % to about 4 wt % of the mascara composition;
the acrylic acid copolymer component is in a range of from about 0.2 wt % to about 5 wt % of the mascara composition;
the eicosene copolymer component is in a range of from about 0.5 wt % to about 3.5 wt % of the mascara composition;
the structurant component is in a range of from about 1 wt % to about 42 wt % of the mascara composition; or
the fiber component is in a range of from about 0.1 wt % to about 0.5 wt % of the mascara composition.
Aspect 3 provides the mascara composition of any one of Aspects 1 or 2, wherein:
the polyacrylate component is in a range of from about 0.25 wt % to about 0.35 wt/o of the mascara composition;
the polyvinylpryrrollidone component is in a range of from about 3.5 wt % to about 4 wt % of the mascara composition;
the acrylate copolymer component is in a range of from about 1.5 wt % to about 2.5 wt % of the mascara composition;
the acrylic acid copolymer component is in a range of from about 0.5 wt % to about 1.5 wt % of the mascara composition;
the eicosene copolymer component is in a range of from about 1.5 wt % to about 2.5 wt % of the mascara composition;
the structurant component is in a range of from about 4 wt % to about 8.5 wt % of the mascara composition; or
the fiber component is in a range of from about 0.1 wt % to about 0.5 wt % of the mascara composition.
Aspect 4 provides the mascara composition of any one of Aspects 1-3, wherein:
the polyacrylate component comprises a mixture of sodium polyacrylate and acrylic acid;
the acrylate copolymer component comprises a mixture of an ammonium acrylate copolymer, water, alcohol denat., disodium deceth-6 sulfosuccinate, laureth-30, and sodium dehydroacetate;
the acrylic acid copolymer component comprises a mixture of water, ethylene/acrylic acid copolymer, Styrene/Acrylates Copolymer, C11-15 pareth-7, C11-15 pareth 40, phenoxyethanol, caprylyl glycol, and sodium laureth-12 sulfate;
the eicosene copolymer comprises an alkylated vinyl pyrrolidone;
the structurant component comprises a paraffin wax, a beeswax, a carnauba wax, or a mixture thereof; or
the fiber component comprises a polyamide fiber, a cellulose fiber, or a mixture thereof.
Aspect 5 provides the mascara composition of Aspect 4, wherein the fiber component comprises a polyamide fiber having length in a range of from about 1 mm to about 5 mm.
Aspect 6 provides the mascara composition of any one of Aspects 4 or 5, wherein the fiber component comprises a polyamide fiber having length in a range of from about 2 mm to about 4 mm.
Aspect 7 provides the mascara composition of any one of Aspects 1-6, wherein the mascara is adapted to dry within a range of from about 1 minute to about 10 minutes following application.
Aspect 8 provides the mascara composition of any one of Aspects 1-7, wherein the mascara is adapted to dry within a range of from about 3 minutes to about 7 minutes following application.
Aspect 9 provides the mascara composition of any one of Aspects 1-8, wherein the mascara visibly lengthens the appearance of an eyelash in a range of from about 20% to about 200%, relative to a baseline eyelash that is free of the mascara.
Aspect 10 provides the mascara composition of any one of Aspects 1-9, wherein the mascara visibly lengthens the appearance of an eyelash in a range of from about 30% to about 50%, relative to a baseline eyelash that is free of the mascara.
Aspect 11 provides the mascara composition of any one of Aspects 1-10, wherein the mascara visibly increases the volume of an eyelash in a range of from about 5× to about 15×, relative to a baseline eyelash that is free of the mascara.
Aspect 12 provides the mascara composition of any one of Aspects 1-11, wherein the mascara visibly increases the volume of an eyelash in a range of from about 6× to about 10×, relative to a baseline eyelash that is free of the mascara.
Aspect 13 provides the mascara composition of any one of Aspects 1-12, wherein the mascara composition has a viscosity in a range of from 900,000 cP to about 9,000,000 cP at a temperature in a range of about 25° C. to about 57° C.
Aspect 14 provides the mascara composition of any one of Aspects 1-13, further comprising a emulsifier, protective agent, or a mixture thereof.
Aspect 15 provides the mascara composition of any one of Aspects 1-14, further comprising a pigment component.

Aspect 16 provides the mascara composition of Aspect 15, wherein the pigment component is in a range of from about 5 wt % to about 15 wt % of the composition.
Aspect 17 provides the mascara composition of any one of Aspects 15 or 16, wherein the pigment component comprises iron oxides, natural aluminum silicates such as ocher, titanium (di) oxide, mica, kaolin, U bra and red bolus, calcium carbonate, talc, mica titanium oxide, mica titanium oxide-iron oxide, carmine, blue #1, yellow #5, chromium oxide, and mixtures thereof.
Aspect 18 provides the mascara composition of any one of Aspects 1-17, further comprising a conditioning agent.
Aspect 19 provides the mascara composition of Aspect 18, wherein the conditioning agent is in a range of from about 0.1 wt % to about 2 wt % of the composition.
Aspect 20 provides the mascara composition of any one of Aspects 18 or 19, wherein the conditioning agent comprises vitamin E.
Aspect 21 provides the mascara composition of any one of Aspects 1-20, further comprising an antioxidant agent.
Aspect 22 provides the mascara composition of Aspect 21, wherein the antioxidant agent is in a range of from about 0.1 wt % to about 2 wt % of the composition.
Aspect 23 provides the mascara composition of any one of Aspects 21 or 22, wherein the antioxidant agent comprises vitamin C, vitamin A, folic acid, tcopheryl acetate, histidine, glycine, tyrosine, tryptophan, carotene, or a mixture thereof.
Aspect 24 provides the mascara composition of any one of Aspects 1-23, further comprising a humectant.
Aspect 25 provides the mascara composition of Aspect 24, wherein the humectant comprises propylene glycol, butylene glycol, glycerol, or a mixture thereof.
Aspect 26 provides a kit comprising:
a vessel;
the mascara composition of any one of Aspects 1-25 disposed within the vessel; and
an applicator in contact with at least a portion of the mascara composition.
Aspect 27 provides a method of making the mascara composition of any one of Aspects 1-26, the method comprising processing the polyacrylate component, the polyvinylpryrrollidone component, the acrylate copolymer component, the acrylic acid copolymer component, the eicosene copolymer component, the structurant component, and, optionally, the fiber component.

1. A mascara composition comprising:
a polyacrylate component;
a polyvinylpryrrollidone component;
an acrylate copolymer component;
an acrylic acid copolymer component;
an eicosene copolymer component; and optionally
a structurant component;
a fiber component; or
a mixture thereof.

2. The mascara composition of claim 1, wherein:
the polyacrylate component is in a range of from about 0.1 wt % to about 1 wt % of the mascara composition;
the polyvinylpryrrollidone component is in a range of from about 1 wt % to about 5 wt % of the mascara composition;
the acrylate copolymer component is in a range of from about 0.5 wt % to about 4 wt % of the mascara composition;
the acrylic acid copolymer component is in a range of from about 0.2 wt % to about 5 wt % of the mascara composition;
the eicosene copolymer component is in a range of from about 0.5 wt % to about 3.5 wt % of the mascara composition;
the structurant component is in a range of from about 1 wt % to about 42 wt % of the mascara composition; or
the fiber component is in a range of from about 0.1 wt % to about 0.5 wt % of the mascara composition.

3. The mascara composition of claim 1, wherein:
the polyacrylate component is in a range of from about 0.25 wt % to about 0.35 wt % of the mascara composition;
the polyvinylpryrrollidone component is in a range of from about 3.5 wt % to about 4 wt % of the mascara composition;
the acrylate copolymer component is in a range of from about 1.5 wt % to about 2.5 wt % of the mascara composition;
the acrylic acid copolymer component is in a range of from about 0.5 wt % to about 1.5 wt % of the mascara composition;
the eicosene copolymer component is in a range of from about 1.5 wt % to about 2.5 wt % of the mascara composition;
the structurant component is in a range of from about 4 wt % to about 8.5 wt % of the mascara composition; or
the fiber component is in a range of from about 0.1 wt % to about 0.5 wt % of the mascara composition.

4. The mascara composition of claim 1, wherein:
the polyacrylate component comprises a mixture of sodium polyacrylate and acrylic acid;
the acrylate copolymer component comprises a mixture of an ammonium acrylate copolymer, water, alcohol denat., disodium deceth-6 sulfosuccinate, laureth-30, and sodium dehydroacetate;
the acrylic acid copolymer component comprises a mixture of water, ethylene/acrylic acid copolymer, Styrene/Acrylates Copolymer, C11-15 pareth-7, C11-15 pareth 40, phenoxyethanol, caprylyl glycol, and sodium laureth-12 sulfate;
the eicosene copolymer comprises an alkylated vinyl pyrrolidone;
the structurant component comprises a paraffin wax, a beeswax, a carnauba wax, or a mixture thereof; or
the fiber component comprises a polyamide fiber, a cellulose fiber, a copolymer of azacyclotridecan-2-one, polymer with hexahydro-2H-azepin-2-one, or a mixture thereof.

5. The mascara composition of claim 4, wherein the fiber component comprises a polyamide fiber having length in a range of from about 1 mm to about 5 mm.

6. The mascara composition of claim 4, wherein the fiber component comprises a polyamide fiber having length in a range of from about 2 mm to about 4 mm.

7. The mascara composition of claim 1, wherein the mascara is adapted to dry within a range of from about 1 minute to about 10 minutes following application.

8. The mascara composition of claim 1, wherein the mascara is adapted to dry within a range of from about 3 minutes to about 7 minutes following application.

9. The mascara composition of claim 1, wherein the mascara visibly lengthens the appearance of an eyelash in a range of from about 20% to about 200%, relative to a baseline eyelash that is free of the mascara.

10. The mascara composition of claim 1, wherein the mascara visibly lengthens the appearance of an eyelash in a range of from about 30% to about 50%, relative to a baseline eyelash that is free of the mascara.

11. The mascara composition of claim 1, wherein the mascara visibly increases the volume of an eyelash in a range of from about 5× to about 15×, relative to a baseline eyelash that is free of the mascara.

12. The mascara composition of claim 1, wherein the mascara visibly increases the volume of an eyelash in a range of from about 6× to about 10×, relative to a baseline eyelash that is free of the mascara.

13. The mascara composition of claim 1, wherein the mascara composition has a viscosity in a range of from 900,000 cP to about 9,000,000 cP at a temperature in a range of about 25° C. to about 57° C.

14. The mascara composition of claim 1, further comprising an emulsifier, protective agent, or a mixture thereof.

15. The mascara composition of claim 1, further comprising a pigment component.

16. The mascara composition of claim 15, wherein the pigment component is in a range of from about 7 wt % to about 15 wt % of the composition.

17. The mascara composition of claim 15, wherein the pigment component comprises iron oxides, natural aluminum silicates such as ocher, titanium (di) oxide, mica, kaolin, U bra and red bolus, calcium carbonate, talc, mica titanium oxide, mica titanium oxide-iron oxide, carmine, blue #1, yellow #5, chromium oxide, and mixtures thereof.

18. The mascara composition of claim 1, further comprising a conditioning agent.

19. The mascara composition of claim 18, wherein the conditioning agent is in a range of from about 0.1 wt % to about 2 wt % of the composition.

20. The mascara composition of claim 1, wherein the conditioning agent comprises vitamin E.

21. (canceled)

22. (canceled)

23. (canceled)

24. (canceled)

25. (canceled)

26. (canceled)

27. (canceled)
